(19) United States
(12) Patent Application Publication
(10) Pub. No.: US 2025/0018177 A1
(43) Pub. Date: Jan. 16, 2025

(54) MEDICAL GUIDE INSTRUMENT, MEDICAL INSTRUMENT SET, MEDICAL DEVICE, AND MEDICAL METHOD

(71) Applicant: Joimax GmbH, Karlsruhe (DE)

(72) Inventors: Wolfgang RIES, Linkenheim (DE); Lars SCHENDZIELORZ, Linkenheim (DE); Jost THISSEN, Duesseldorf (DE)

(73) Assignee: Joimax GmbH, Karlsruhe (DE)

(21) Appl. No.: 18/901,986

(22) Filed: Sep. 30, 2024

Related U.S. Application Data
(63) Continuation of application No. PCT/EP2023/057809, filed on Mar. 27, 2023.

(30) Foreign Application Priority Data
Mar. 30, 2022 (DE) 10 2022 107 541.9

(51) Int. Cl. A61N 1/05; A61B 1/00; A61B 1/018

(52) U.S. Cl. CPC A61N 1/0551; A61B 1/00154; A61B 1/018

(57) ABSTRACT

A medical guide instrument for a medical electrode in order to position the latter against spinal nerves in a spinal canal. The guide instrument has a distal feed tube for receiving the electrode, wherein when the guide instrument is in an insertion position a first portion of the feed tube has a curvature, while when the guide instrument is in a feed position the first portion of the feed tube is substantially linear. An instrument set having the guide instrument and an electrode, a medical device having the guide instrument and an endoscope, and a method for positioning the medical electrode against spinal nerves are also provided.

This nonprovisional application is a continuation of International Application No. PCT/EP2023/057809, which was filed on Mar. 27, 2023, and which claims priority to German Patent Application No. 10 2022 107 541.9, which was filed in Germany on Mar. 30, 2022, and which are both herein incorporated by reference.

### BACKGROUND OF THE INVENTION

### Field of the Invention

The invention relates to a medical guide instrument for a medical electrode for positioning the electrode on spinal nerves in a spinal canal, in particular on spinal nerve nodes. Furthermore, the invention relates to a medical instrument set, a medical device, and a medical method.

### Description of the Background Art

Spinal cord stimulation has become an important pillar of chronic pain therapy in recent decades. From this, the stimulation of the dorsal root ganglia, i.e., of spinal nerve nodes, has emerged as a new technique for the treatment of, in particular monoradicular, pain in the upper and lower extremities, in particular of peripheral origin. Stimulation of the dorsal root ganglion is a new, effective form of therapy for pain conditions that affect one or more different nerve roots.

Common methods involve implanting the electrode in the patient's spinal canal via the intralaminar access. The electrodes are inserted into the patient's spinal canal (epidural) up to the desired dorsal root ganglion. However, this is often difficult in patients with epidural scarring, which blocks movement of the electrode and often causes undesirable distraction, especially since the electrode is designed to be flexible due to its use for contact to the dorsal root ganglion. In particular for the aforementioned patient group, reliable and rapid placement of the electrode at the desired location is not possible using current devices and methods.

### SUMMARY OF THE INVENTION

It is therefore an object of the invention to eliminate the disadvantages of the prior art and, in particular, to allow for better positioning of the electrode.

The object of the invention is achieved, in an example, with a medical guide instrument that has a distal feed tube for receiving the electrode, wherein when the guide instrument is in an insertion position a first portion of the feed tube has a curvature, and wherein when the guide instrument is in a feed position the first portion of the feed tube is substantially linear.

The object of the invention is further achieved with a medical instrument set with a medical guide instrument according to the invention and with a medical electrode with at least one electrically conductive contact surface and with a line connected to the electrode. Furthermore, the invention is achieved with a medical device with a medical guide instrument according to the invention and with a medical endoscope, wherein the guide instrument can be received in a working canal of the endoscope.

Also, the invention is achieved with a medical method for positioning a medical electrode on spinal nerves with the aid of a medical guide instrument according to the invention with the following steps: providing access to the spinal nerves, inserting the medical guide instrument in the feed position for positioning the electrode on the spinal nerves, triggering the insertion position of the medical guide instrument and inserting the electrode through the medical guide instrument through its feed tube until the electrode is arranged on the nerves.

The invention is based on the fundamental idea that the guide instrument according to the invention ensures reliable guidance of the medical electrode, in particular within the patient's body during the surgical procedure. In particular, epidural scar tissue of the patient, which often causes an undesirable deflection of the electrode with the known guide instruments, can be overcome by the defined guidance of the electrode by the guide instrument according to the invention in the insertion position, so that the electrode reaches its intended location safely and quickly and the planned pain therapy can be continued. In the sense of the invention, the insertion position of the guide instrument refers to a position in which the electrode can be pushed through the guide instrument and reach a predefined location. In the sense of the invention, this is achieved by the feed tube being curved at its first portion. In the feed position, the guide instrument can be particularly easily positioned at a predetermined location, in particular due to the linear design of the feed tube in the feed position.

In the feed position, the first portion of the feed tube can be designed coaxially, wherein coaxial in the sense of the invention refers to the (axial) extension direction of the feed instrument according to the invention. In the sense of the invention, a distal direction points towards the patient, a proximal direction towards the user/surgeon. In the sense of an imaginary three-dimensional cylindrical coordinate system, an axial direction is aligned with the extension direction of the feed instrument, while a radial direction is perpendicular to the axial direction, as is a circumferential direction.

The feed tube of the guide instrument can be designed as an, in particular flexible, (hollow) catheter, with at least one (hollow) cylindrical portion.

In the insertion position of the guide instrument, the curvature of the first portion of the feed tube can be user-defined, in particular user-defined variably, preferably manually adjustable. The curvature of the feed tube in the insertion position of the guide instrument can have a predefined axis of curvature and/or a predefined curvature radius, wherein the curvature radius is in particular between 6 mm and 15 mm, preferably between 9 mm and 12 mm. In particular, the axis of curvature is perpendicular to the axial direction and/or is arranged in a horizontal plane.

The first portion can be arranged in a distal region of the feed tube, for example as a distal end region, in order to specifically deflect the electrode as immediately as possible after it emerges from the guide instrument. The distal end region is preferably designed to be flexible.

A change between the feed position and the insertion position of the guide instrument can be carried out due to an, in particular linear, displacement of the feed tube in order to change between the feed position and the insertion position of the guide instrument in a structurally simple manner.

The guide instrument preferably has at least one actuator by means of which the insertion position and in particular the feed position of the guide instrument can be assumed. To facilitate operability, the actuator preferably has at least partial corrugation on a surface facing the user. The actuator can be designed to be manually operable and/or linearly movable and/or rotatable. The feed tube can be linearly movable by means of an actuator in order to be able to assume its insertion position and/or its feed position.

The actuator can be designed as a slide, in particular as a linear slide, in order to allow for a particularly simple change from the feed position of the guide instrument to its insertion position. For this purpose, the actuator can be guided as a slide in a recess in the feed tube assigned to it, which is designed in particular as an elongated hole. Preferably, the actuator is at least indirectly connected to the feed tube, in particular directly and/or in a positive fit, so that in particular an axial movement of the feed tube can take place by actuating the actuator. For the, in particular direct, positive connection of the actuator to the feed tube, the actuator preferably has at least one projection directed towards the feed tube, which engages in a positive fit in at least one particularly radial recess of the feed tube. Alternatively, the actuator may have a recess into which a projection of the feed tube directed towards the recess engages.

The actuator can be associated with a guide part which is in particular connected in a positive fit, in particular screwed, to a proximal handle part. By the example of the guide part and the handle part, a functional-spatial division of the guide instrument can be realized, in which, in the sense of the invention, the handling of the guide instrument can be carried out by the handle part and the actuation of the insertion position of the guide instrument can be carried out by the guide part. The handle part can be detachably connected to the guide part.

Preferably, the feed tube has at least one linear, in particular coaxial portion, wherein the length of the linear portion is in particular between 250 mm and 350 mm. The linear portion is, in particular rigid, so that it is linear even and especially in the insertion position of the guide instrument. Alternatively, the linear portion can be designed flexibly. Preferably, the feed tube is formed in one piece. The feed tube can be formed of a plurality of components, which are, however, connected in one piece, in particular welded together.

The feed tube can be designed in several parts, in particular in two parts, wherein a first distal part of the feed tube can comprise the first portion that can be bent in the sense of the invention. A second, proximal part of the feed tube may in particular comprise the linear portion which extends, for example, over at least 75% of the total length of the feed tube. The first part of the guide tube can be connected in a positive fit to its second part, for example by means of an, in particular rectangular, recess in the first part, into which a complementarily shaped elevation of the second part engages, so that a positive connection is formed between the first part and the second part of the feed tube along the extension direction of the feed tube. In the sense of the invention, the first part of the feed tube can also be welded to its second part in one piece. The second part of the feed tube can in particular have the above-mentioned recess for the positive connection with the actuator. At least a part of the feed tube may include or consist of a component made of plastics material.

The guide instrument can be in the insertion position independently without any external force. This is achieved, for example, in that the feed tube has, at least in portions, in particular in the region of the first region, at least one component made of a resilient material, such as a shape memory alloy, in particular a nickel-titanium alloy, for example nitinol, and/or a plastics material, in particular polyethylene (PE).

The feed tube can be surrounded at least in sections, in particular in a distal region, by a particularly rigid guide tube, wherein the guide tube is designed, for example, as a hollow cylinder and/or is aligned parallel to the feed tube, in particular coaxially thereto. The length of the guide tube corresponds in particular to the length of the linear portion of the feed tube, wherein the axial length of the guide tube is preferably between 250 mm and 350 mm. The feed tube and/or the guide tube preferably has a circular cross section with in particular a diameter between 3 mm and 4 mm, preferably 3.5 mm. In an advantageous development of the invention, the feed tube is arranged with a tight fit within the guide tube, wherein the feed tube is, in particular axially, movable relative to the guide tube. The feed tube can at least partially comprise components made of metal, preferably stainless steel, and/or a resilient material, preferably plastics material.

To guide the medical electrode, the guide instrument can have an inner bore for receiving the electrode, wherein the inner bore extends in particular over the entire axial length of the guide instrument. The inner bore is, in particular coaxially, aligned. The guide instrument is designed, in particular, to be reusable or, alternatively, as a disposable instrument. In particular, if the guide instrument is designed as a disposable instrument, the feed tube can be made of plastics material.

The feed tube can be connected to the actuator at least indirectly via a pull wire. For example, a proximal end region of the feed tube and/or an upper side of the feed tube is connected to the pull wire. The pull wire can be connected to an abutment. Preferably, the actuator, which is designed in particular as a slide, is connected to an abutment.

The feed tube and/or the guide tube can have an elliptical cross section, wherein the diameter of the feed tube and/or the diameter of the guide tube in a first semi-axis of the cross section can be between 2.5 mm and 3.5 mm and in a second semi-axis of the cross section can be between 1.5 mm and 2 mm, in particular 1.7 mm. The guide instrument can have a flattening on its outer surface, in particular in a region facing the actuator, which extends in particular over an azimuthal angle range between 30° and 90°. The flattening creates a defined position of the guide region when it is placed on an operating table, for example.

This prevents accidental dropping and thus contamination of the guide instrument.

The guide instrument can be designed as a disposable instrument. The first portion of the feed tube can be arranged in an axially central portion so that an improved guiding of the electrode in the spinal canal of the patient is possible.

Preferably, an adapter part with an inner bore for receiving the electrode is provided in order to simplify the insertion of the electrode into the guide instrument. Further preferably, the feed tube extends at least partially through the adapter part in order to further improve the insertion of the electrode. The inner bore in particular has a circular cross section and/or is coaxially aligned. The adapter part is preferably positioned in a proximal region of the guide instrument or in the distal guide part of the guide instrument. The guide part may have a handle to improve the operability of the guide instrument. The adapter part can in particular be arranged at the axial height of the handle part.

Preferably, the inner bore of the adapter part can be radially widened towards the proximal end face and/or the distal end face of the adapter part. In particular, the inner bore can be continuously widened towards one of the end faces, resulting, for example, in a funnel-shaped configuration of the inner bore.

The inner bore of the adapter part preferably can have a lateral connection piece arranged at a finite angle to the longitudinal axis of the guide instrument with an inner bore for receiving the electrode, wherein the inner bore of the connection piece is connected to the inner bore of the adapter part. In this way, for example, the electrode can be introduced into the inner bore of the adapter part at a finite angle other than 0°, so that the axial view of the guide instrument is not impaired by the electrode. For example, at least one component of the guide instrument is X-ray visible in order to be able to determine the current position of the guide instrument during the surgical procedure. The guide instrument, in particular a body part thereof, can have at least one preferably coaxially aligned bore in order to reduce the weight of the guide instrument according to the invention and to ensure its easier applicability, in particular when used over a longer period of time.

The at least one electrically conductive contact surface of the electrode of the instrument set can be formed in particular on a distal region of the electrode. The electrode is preferably cylindrical and has in particular at least two, preferably eight contact surfaces, which are arranged axially one behind the other, for example. The electrode can have a maximum of 16 contact surfaces. An electrically non-conductive insulating surface can be arranged between two electrically conductive contact surfaces to prevent short circuits. At least one, in particular distal, portion of the line electrically connected to the electrode can be designed to be elastic and/or at least one portion of the line can be designed to be rigid. In this way, the electrode and/or the line electrically connected to the electrode can adapt in particular to the first portion of the feed tube which is curved in the insertion position of the guide instrument. The electrode and/or the electrical line has a cylindrical shape with an outer diameter of, for example, between 0.5 mm and 1.5 mm. The length of the line is preferably between 400 mm and 800 mm. The electrode can be connected to an electrical pulse generator, wherein in particular the contact surfaces of the electrode can be controlled independently of one another by the pulse generator.

The medical device comprises in particular the medical instrument set with the medical guide instrument according to the invention and/or can be further developed such that the guide instrument, in particular its feed tube and/or the guide tube of the guide instrument, can be accommodated in the working canal of the endoscope. The endoscope may have a flushing connector and/or an optical imaging device, in particular a camera. In particular, the endoscope is designed as a disposable instrument and/or has at least one disposable component. Further, the endoscope can be designed to be reusable.

The method according to the invention can be carried out with a medical guide instrument according to the invention and/or with a medical instrument set according to the invention and/or with a medical device according to the invention. In a further development of the method according to the invention, the access to the spinal nerves, in particular epidural, takes place transforaminally, i.e., through an intervertebral foramen as a (lateral) intervertebral foramen between two adjacent vertebral bodies and/or intralaminarly, i.e., through an intralaminar window at the back (anterior) between two adjacent vertebral bodies.

The medical guide instrument can be inserted by means of inserting a medical device according to the invention. In addition, the medical guide instrument can be inserted under visual inspection and/or X-ray control.

The electrode can be placed on a nerve ganglion, in particular on a dorsal nerve ganglion of the spinal nerves, to ensure effective application of current pulses. After insertion, the electrode can be connected to an electrical pulse generator and, in particular after insertion, the remaining components of the medical guide instrument and/or medical device can be removed from the surgical site. Preferably, the electrode can be arranged on the spinal nerves, for example connectible by means of loops and/or anchor elements.

Further scope of applicability of the present invention will become apparent from the detailed description given hereinafter. However, it should be understood that the detailed description and specific examples, while indicating preferred embodiments of the invention, are given by way of illustration only, since various changes, combinations, and modifications within the spirit and scope of the invention will become apparent to those skilled in the art from this detailed description.

### BRIEF DESCRIPTION OF THE DRAWINGS

The present invention will become more fully understood from the detailed description given hereinbelow and the accompanying drawings which are given by way of illustration only, and thus, are not limitive of the present invention, and wherein:

FIG. 1 is a side view of an example of a guide instrument according to the invention;

FIG. 2 is a plan view of the guide instrument in accordance with FIG. 1;

FIG. 3 is an axial longitudinal section of the guide instrument in accordance with FIG. 1;

FIG. 4 is a cross section of the guide instrument in accordance with FIG. 1;

FIG. 5 is a detailed view of FIG. 4;

FIG. 6 is an enlarged view of a feed tube,

FIG. 7 shows the guide instrument in accordance with FIG. 1 in a feed position,

FIG. 8 shows the guide instrument in accordance with FIG. 7 in an insertion position,

FIG. 9 shows the guide instrument in accordance with FIG. 8 with an inserted electrode,

FIG. 10, 11 shows a further configuration of the feed tube in an insertion position,

FIG. 12 shows a further configuration of the feed tube in an insertion position,

FIG. 13 is a complete representation of the electrode of FIG. 9,

FIG. 14 shows the guide instrument of FIG. 9 in an insertion position with inserted electrode and with an endoscope,

FIG. 15 is an enlarged view of FIG. 14 in the region of the distal feed tube,

FIG. 16 is a perspective proximal view of the guide instrument with endoscope in accordance with FIG. 14,

FIG. 17 is a side view of the guide instrument according to the invention in an example,

FIG. 18 is a plan view of the guide instrument in accordance with FIG. 17;

FIG. 19 is an axial longitudinal section of the guide instrument in accordance with FIG. 17;

FIG. 20 is a perspective view from distal below of the guide instrument in accordance with FIG. 17,

FIG. 21 shows the guide instrument in accordance with FIG. 17 in a feed position,

FIG. 22 shows the guide instrument in accordance with FIG. 21 in an insertion position with the electrode to be inserted;

FIG. 23 shows the guide instrument in accordance with FIG. 22 with inserted electrode,

FIG. 24 shows the guide instrument in accordance with FIG. 17 in an insertion position with an endoscope,

FIG. 25 is an enlarged view of the guide instrument in accordance with FIG. 24 in the region of a distal feed tube,

FIG. 26 is a perspective proximal view of the guide instrument with endoscope in accordance with FIG. 24,

FIG. 27 is a side view of an example of the guide instrument according to the invention with endoscope,

FIG. 28 is a perspective proximal view of the guide instrument with endoscope in accordance with FIG. 27, and

FIG. 29 is a proximal view of an implanted electrode with pulse generator.

### DETAILED DESCRIPTION

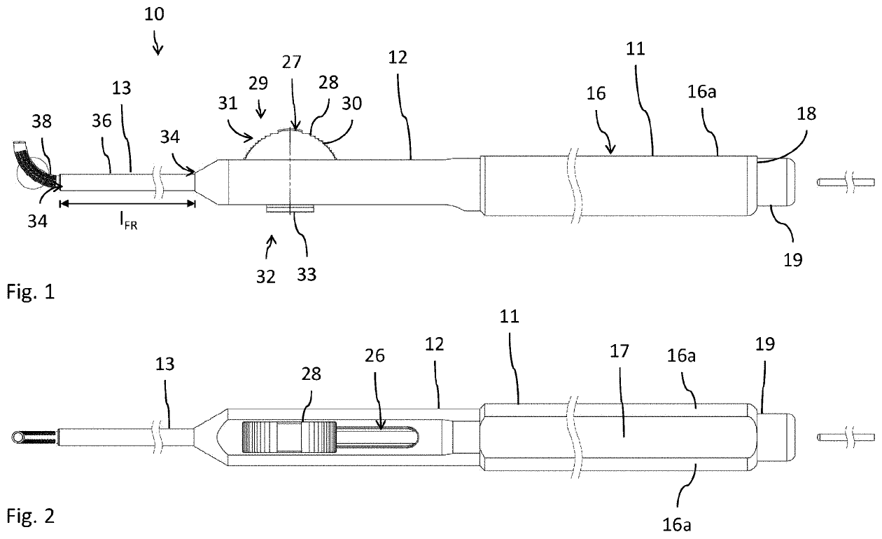

FIG. 1 shows a side view of a guide instrument 10 according to the invention in an example with a proximal body part 11, an axially central handle part 12 and a distal guide part 13.

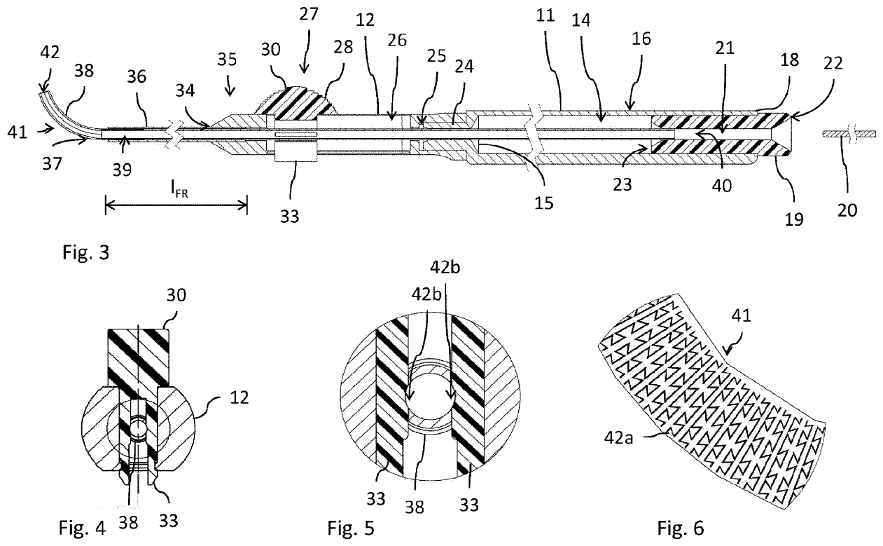

The proximal body part 11 is substantially designed as an axially aligned hollow cylinder with an inner bore 14 and an axial base 15 arranged within it, which is shown in particular in the longitudinal section of FIG. 3. On its upper surface 16, the body part 11 in accordance with FIG. 2 has a recess 17 with a partially circular cross section, which is surrounded on both sides by a flattening 16a with a flat surface, so that the guide instrument 10 does not fall uncontrollably from an instrument table after it has been put down, but remains in a defined position. The body part 11 is open towards its proximal end face 18 so that an adapter part 19 for introducing an electrode 20 can be inserted into the inner bore 14 and connected to the body part 11.

In the connected state, the adapter part 19 engages in the proximal bore 14 of the body part 11 and projects proximally beyond it. The distal outer diameter of the adapter part 19 is smaller than the inner diameter of the body part 11. The adapter part 19 has an axially continuous inner bore 21 which is radially widened towards the proximal end face 22 of the adapter part 19 in order to facilitate the insertion of the electrode 20. The inner bore 21 of the adapter part 19 is also widened towards its distal end face 23 to facilitate the insertion of a feed tube 38, which is described further below. In the example shown, the electrode 20 has an outer diameter of 1.4 mm. The diameter of the inner bore 21 is slightly larger than the diameter of the electrode 20 to be inserted, so that the latter can penetrate the inner bore 21 and is freely axially movable within it, which is also described below.

The proximal body part 11 of the guide instrument 10 is connected in the distal direction to the handle part 12, wherein a distal, cylindrical pin 24 of the body part 11 engages in an inner bore 25 of the handle part 12. The handle part 12 is, similarly to the body part 11, designed as an axially aligned cylinder with an elongated hole-shaped recess 26 facing upwards in accordance with the plan view of FIG. 2, which opens into the inner bore 25 of the handle part 12. The recess 26 serves as a guide for an actuator 27 which is arranged partially within the recess 26 and which, in the example in accordance with FIGS. 1 to 6, is designed as a manually operable linear slide 28 as a slider in order to bring the guide instrument 10 in particular from a feed position into an insertion position, which is described further below. The linear slide 28 has a semicircular sliding button 30 with a grooved surface 31 on its upper side 29 shown in FIG. 1 in order to improve operability. On its underside 32 in accordance with FIG. 1, the linear slide 28 has two rectangular projections 33 shown in the longitudinal section of FIG. 3, which pass through the recess 26 and the inner bore 25 of the handle part 12 in such a way that the projections 33 protrude beyond the handle part 12, see FIGS. 1 and 3. The cross section of the handle part 12 tapers towards its distal end face 34, while the cross section of the inner bore 25 in the distal region 35 of the handle part 12 remains unchanged.

The distal guide part 13 has a rigid guide tube 36 made of stainless steel, which, coming from the distal direction, is inserted into the inner bore 25 of the handle part 12 and is connected thereto. In the example shown of the guide instrument 10 in accordance with FIGS. 1 to 6, the region of the guide tube 36 projecting distally beyond the handle part has an axial length IFR between 250 mm to 350 mm, measured from the distal end face 34 of the handle part 12 to the distal end face 37 of the guide tube 36. The guide tube 36 is designed as a hollow cylinder with a circular cross section, wherein the diameter of the guide tube 36 is 3.50 mm and remains unchanged over its axial length.

In particular in accordance with the section of FIG. 3, a feed tube 38 is arranged within the guide tube 36, the axial length of which is greater than that of the rigid guide tube 36. The feed tube 38 engages in an interior space 39 of the guide tube 36, the inner bore 25 of the handle part 12, the bore 14 of the body part 11, and the inner bore 21 of the adapter part 19, wherein, in the insertion position of the guide instrument 10 shown in FIG. 3, the proximal end face 40 of the feed tube 38 is arranged within the inner bore 21 of the adapter part 19 and does not completely penetrate it. The outer diameter of the feed tube substantially corresponds to the inner diameter of the inner bore 21 of the adapter part 19, so that the feed tube 38 is arranged with a tight fit within the adapter part 19, but is axially movable there.

In particular, at its distal end region 41, the feed tube 38 has components made of a shape memory alloy, so that the distal end region 41 of the feed tube 38 is designed to be flexible. In the example of FIGS. 1 to 6, which show the guide instrument according to the invention in the insertion position, the distal end region 41 of the feed tube 38 has a curvature directed upwards in FIG. 1, which is approximately 90° relative to the orientation of the guide tube 36, so that the distal end face 42 of the feed tube 38 is deflected upwards by approximately 9 mm to 12 mm from the lower, distal end face 37 of the guide tube 36. This corresponds to a curvature radius of 9 mm to 12 mm. In this respect, in the example shown, the curved distal end region 41 of the feed tube 38 corresponds to its first portion 41 in the sense of the invention. FIG. 6 shows a detailed view of the flexible distal end region 41 of the feed tube 38. Accordingly, the region 41 shown is provided with a plurality of recesses 42a, which allow for a resilient deformation of the feed tube 38, here: the curvature shown, in the insertion position. The inner diameter of the feed tube 38 is slightly larger than 1.40 mm, so that the electrode 20 with its outer diameter of 1.40 mm can be pushed axially through the feed tube 38 and is axially movable within the feed tube 38.

In particular, in accordance with FIGS. 3 to 5, it can be seen that the projections 33 of the sliding button 30, which are directed downwards, engage in axially aligned, lateral recesses 42b of the feed tube 38, so that the sliding button 30, and thus the linear slide 28 as a whole, are connected in a positive fit to the feed tube 38 in the axial direction in the sense of a quickly removable locking connection. FIG. 4 shows a cross section through the guide instrument 10 in accordance with FIG. 3 at the axial height of the linear slide 28. FIG. 5 is a radially centered detail view of FIG. 4. The projections 33 of the sliding button 30, which in particular pass through the recess of the handle part 26, are each subjected to a force directed laterally outwards and thus serve as snap hooks with which the sliding button 30 is connected to the handle part 12 in a clamping, i.e., force-fitting manner.

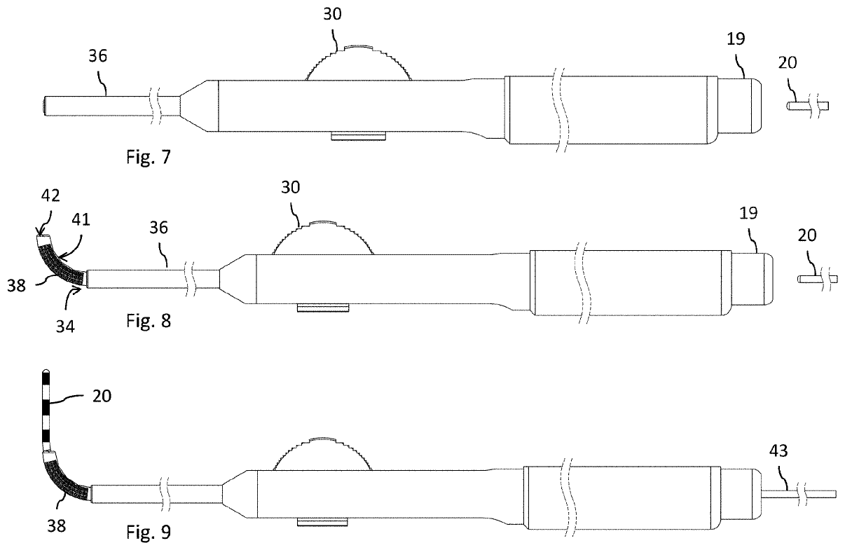

If the sliding button 30 is moved by a user in the proximal direction starting from the position shown in accordance with FIG. 3, the feed tube 38 follows this axial movement. As a result, the distal end face 42 of the feed tube 38 is retracted into the guide tube 36 so that the feed tube 38, due to its geometry and its resilient design, is arranged completely and coaxially within the guide tube 36. The proximal end face 40 of the feed tube 38 remains within the inner bore 21 of the adapter part 19. This configuration corresponds to the feed position of the guide instrument 10 according to the invention and is shown in FIG. 7.

If the sliding button 30 is now moved in the distal direction, starting from the feed position of the guide instrument 10 in accordance with FIG. 7, the resilient feed tube 38 passes through the distal end face 34 of the rigid guide tube 36 and, after emerging from the guide tube 36, assumes its already described curvature due to the shape memory alloy of the distal end region 41, see FIG. 8. This configuration corresponds to the insertion position of the guide instrument 10. If, in the insertion position of the guide instrument 10, the electrode 20 with an electrical line 43 connected thereto is introduced from the proximal side through the adapter part 19 into the interior 39 of the feed tube 38 and moved further distally, the electrode 20 finally penetrates the distal end face 42 of the feed tube 38, which is angled relative to the axial direction due to the curvature, here at an angle of approximately 90°. This makes it possible to transport the flexible electrode 20 to a desired location, which is not possible or only very difficult with the known rigid and linear instruments.

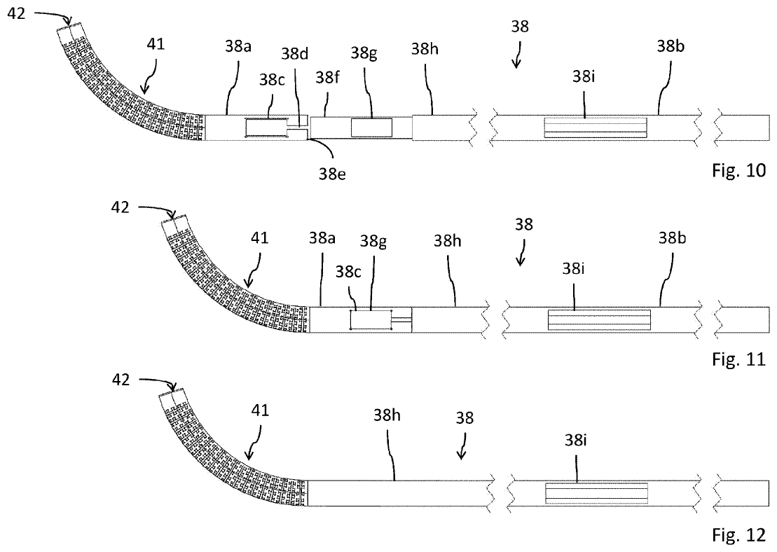

FIG. 10 shows a two-part design of the feed tube 38, which has a first, distal part 38a and a second, proximal part 38b. The first part 38a is shown separately from the second part 38b in FIG. 10. The first part 38a has, as already described, the curved first portion in the distal end region 41, which is provided with several material structures by means of a laser cutting process. Since in FIG. 10 no external forces act on the distal end region 41 of the feed tube 38 shown, the latter, as also already described, automatically assumes the curved position. The first part 38a is provided proximally with a rectangular recess 38c shown in FIG. 10 in the sense of an opening. An axial, milled slot 38d extends proximally from the recess 38c and has a smaller lateral extent than the recess 38c. The slot 38d extends from the rectangular recess 38c to a proximal end face 38e of the first part 38a. The first part 38a is made as a hollow cylinder at least in a region from the recess 38c to its proximal end face 38e.

The second part 38b of the feed tube 38 has on its distal side a coaxially aligned, cylindrical extension 38f which has a smaller diameter than the inner diameter of the first part 38a. The second part 38a can therefore be inserted with its extension 38f into the first part 38a. For the positive connection of the second part 38b with the first part 38a, the extension 38f has a rectangular projection 38g in the sense of an elevation, which is designed complementarily to the recess 38c of the first part 38a. The extension 38f is formed integrally with a body part 38h, the diameter of which corresponds to the diameter of the first part 38a. The body part 38h has a recess 38i in its proximal region through which the rectangular projections 33 of the actuator 27 can engage in order to connect the actuator 27 in a positive fit to the feed tube 38. As the second part 38b is pushed in the direction of the first part 38a, starting from the representation in accordance with FIG. 10, the extension 38f penetrates the first part 38a until the projection 38g is at the same axial height as the recess 38c, so that the former penetrates the latter. The material weakening in the form of the slot 38d of the first part 38a facilitates the insertion of the parts 38a, 38b into one another. The positive engagement of the projection 38g in the recess 38c creates a removable connection between the two parts 38a, 38b in the sense of a locking connection, so that the feed tube 38 as a whole is movable, in particular axially. This state is shown in FIG. 11. FIGS. 10 and 11 substantially show the implementation of the connection of the parts 38a, 38b by means of positive fit. The positive connection is secured by the guide tube 36 in such a way that, after the feed tube 38 has been inserted into the guide tube 36, a reliable connection is ensured during use, even under mechanical stress.

FIG. 12 shows an example of the feed tube 38 which is designed in one piece. This eliminates the need to connect two independent parts 38a, 38b to each other, which results in a structurally simple design. The connection of the feed tube 38 with the guide tube 36 pushed over it is made by means of the recess 38i, as already described above.

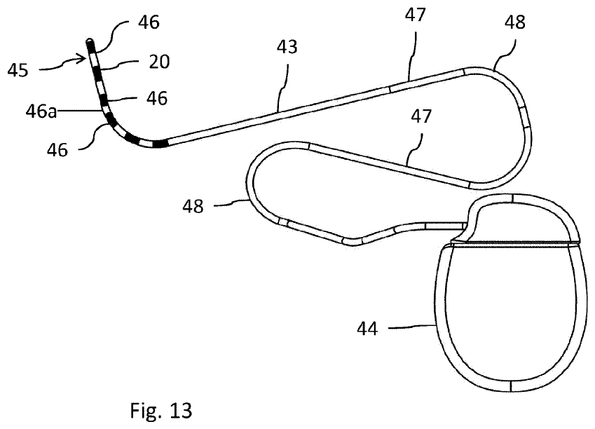

FIG. 13 shows the electrode 20 with the electrical line 43 connected proximally to the electrode 20 and an electrical pulse generator 44 connected to the line, here a pacemaker, which provides the electrical energy to control the electrode 20. In the example of FIG. 13, the electrode 20 is cylindrical and has on its outer surface 45 six electrically conductive contact surfaces 46 arranged axially one behind the other, by means of which the current pulses can be applied in a spatially targeted manner. An electrically non-conductive insulation 46a is provided between each two contact surfaces 46. Each contact surface 46 can be controlled separately by the pulse generator 44, wherein the current pulses are predefined in terms of amplitude and frequency. The electrode 20 is resilient and is shown in a curved arrangement in FIG. 13. In the example of FIG. 13, the electrode 20 is shown in a flexible configuration.

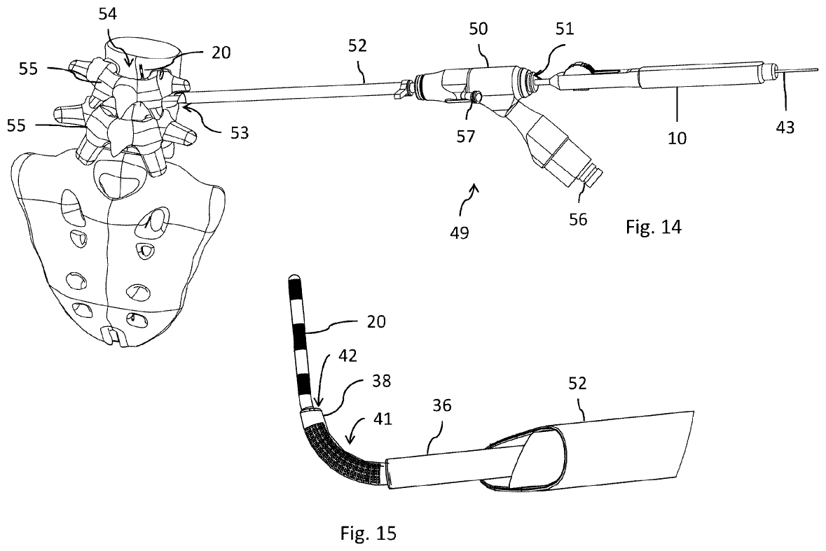

FIG. 14 shows a medical device 49 with the guide instrument 10 according to the invention substantially in the example in accordance with FIGS. 1 and 2, an electrode 20 introduced therein together with an electrical line 43 and with an endoscope 50 in surgical use. Accordingly, the guide tube 36 of the guide instrument 10 is introduced into a coaxially aligned working canal 51 of the endoscope 50. The endoscope 50 accesses the spinal canal 54 of a patient via a distal, rigid working sleeve 52 of the endoscope 50, wherein the working sleeve 52 does not protrude distally beyond the guide tube 36 and the feed tube 38, together with the electrode 20. In FIG. 14, the guide tube 36 of the guide instrument 10, together with the feed tube 38, is introduced through an intervertebral foramen 53, i.e., an intervertebral foramen as an access to the spinal canal 54 of the patient between two adjacent vertebral bodies, here two lumbar vertebral bodies 55, in order to bring the electrode 20 into contact with a dorsal root ganglion within the spinal canal 54. Unlike the example in accordance with FIGS. 1 and 2, the body part 11 of the guide instrument does not have a recess 17, but only the flattening 16a on its upper side.

The guide instrument 10 is in the insertion position in FIG. 14, so that the distal end region 41 of the feed tube 38 is curved in the manner already described, which is shown in particular in accordance with the detailed view of FIG. 15. The electrode 20 passing through the feed tube 38 is deflected according to the curved distal end region 41. In addition, in accordance with FIG. 15, the working sleeve 52 is designed to be distally cannulated. In accordance with FIG. 14, the endoscope 50 has a lateral flushing connector 57 and a lateral optical output 56, wherein the surgical area can be visually viewed by means of said output.

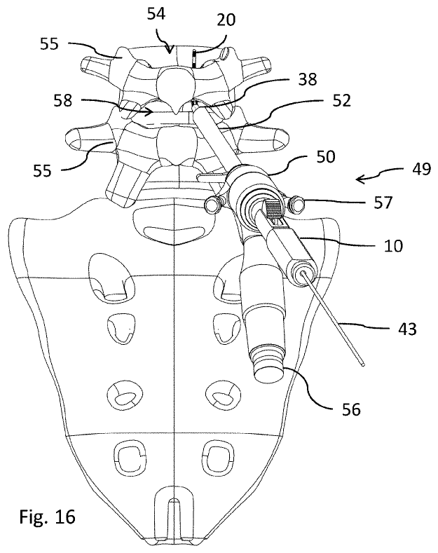

FIG. 16 shows the medical device 49 of FIG. 14 in a perspective, cranioproximal view, wherein access to the spinal canal 54 takes place via an intralaminar window 58 between the two lumbar vertebral bodies 55. In FIG. 16, the guide instrument 10 is also in the insertion position; the electrode 20 is curved upwards by approximately 90° in the spinal canal 54 and is in contact with the dorsal nerve ganglion.

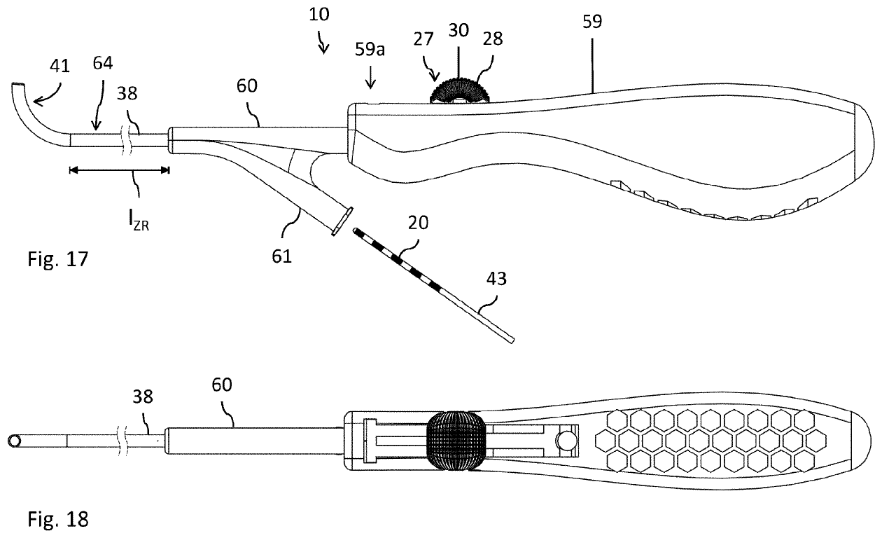

FIG. 17 shows an example of the guide instrument 10 according to the invention, which is designed as a disposable instrument for single use. The guide instrument 10 has a proximal handle part 59 which is connected in a positive fit to a distal guide part 60. The proximal, outer contour of the handle part 59 is designed to be securely enclosed and held by the hand of a user, even and especially during operation. The actuator 27 is provided in a distal region 59a of the handle part 59 and, similarly to the first example of the guide instrument 10, is designed as a linear slide 28 with a sliding button 30, wherein the sliding button 30 is also provided with a profile here.

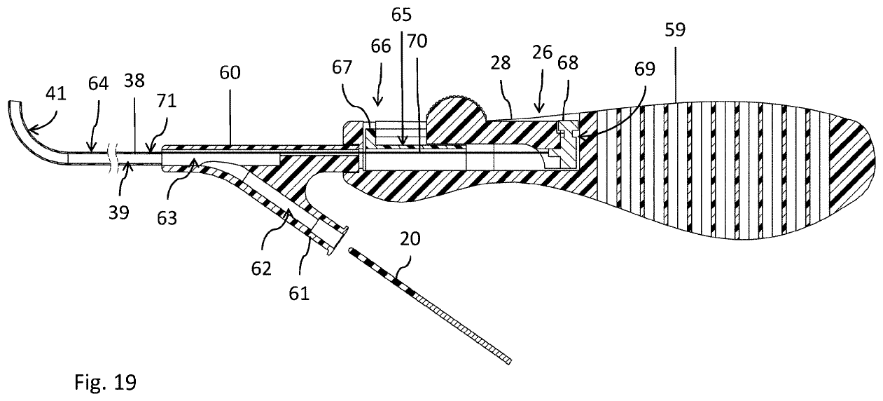

In accordance with FIGS. 17 to 20, the linear slide 28 is in a proximal position which corresponds to the insertion position of the guide instrument 10, in which the first distal portion 41 of the feed tube 38 is angled. In accordance with FIG. 17, the distal guide part 60 has a connection piece 61 pointing downwards to the right with an inner bore 62 which serves as a lateral supply canal, which is also evident from the axial longitudinal section of FIG. 19. The diameter of the inner bore 62 is designed such that an electrode 20 shown in FIG. 17 can be inserted into the inner bore 62 with lateral play, see also FIG. 19. The inner bore 62 of the connection piece 61 is connected to a coaxial inner bore 63 of the guide part 60, so that the electrode 20 can finally be inserted via the inner bores 62, 63 into the interior 39 of the feed tube 38, wherein the feed tube 38 is connected to the guide part 60.

The feed tube 38 is elliptical in cross section and has a diameter of between 2.50 mm and 3.50 mm in the lateral, horizontal direction, corresponding to a first semi-axis, and a diameter of 1.70 mm in the lateral, vertical direction, corresponding to a second semi-axis. In this example, the feed tube 38 has a second, rigid portion 64 with an axial length I_{ZR} between 250 and 350 mm and the already described first curved portion 41 with a curvature of approximately 90°.

From the longitudinal section of the guide instrument 10 in accordance with FIG. 19, it can be seen that the linear slide 28 is mounted in a linearly movable manner on a distal contact surface 65 of the handle part 59. The contact surface 65 has a stop 67 at its distal end region 66 for the distal movement of the linear slide 28. Proximally, the linear slide 28 in accordance with FIG. 19 is in contact with an abutment 68 which in turn is in contact with a proximal end face 69 of the recess 26 of the handle part 59. The abutment 68 is connected to a coaxially aligned pull wire 70 which passes through the recess 26 of the handle part 59 and the inner bore 63 of the guide part 60 and is connected to an upper side 71 of the feed tube 38.

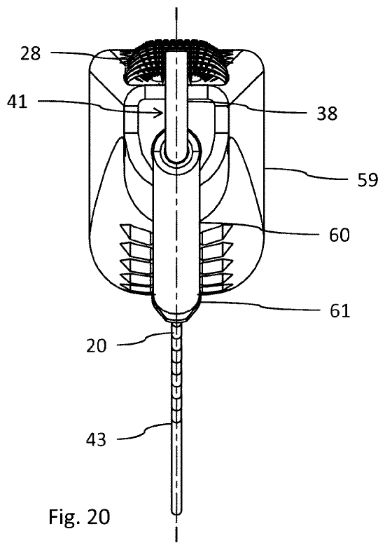

If the linear slide 28 is moved by a user from a distal position in a proximal direction, the resulting pull on the pull wire 70 causes a deflection of the upper side 71 of the feed tube 38 connected to the pull wire 70, so that its distal end region 41 assumes the curved position shown in FIG. 19. In this position, an electrode 20 inserted into the lateral inner bore 62 of the guide part 60 is deflected accordingly during a further axial movement through the distal end region 41 of the feed tube 38. FIG. 20 shows the guide instrument 10 of FIG. 19 in the insertion position in a disto-caudal view.

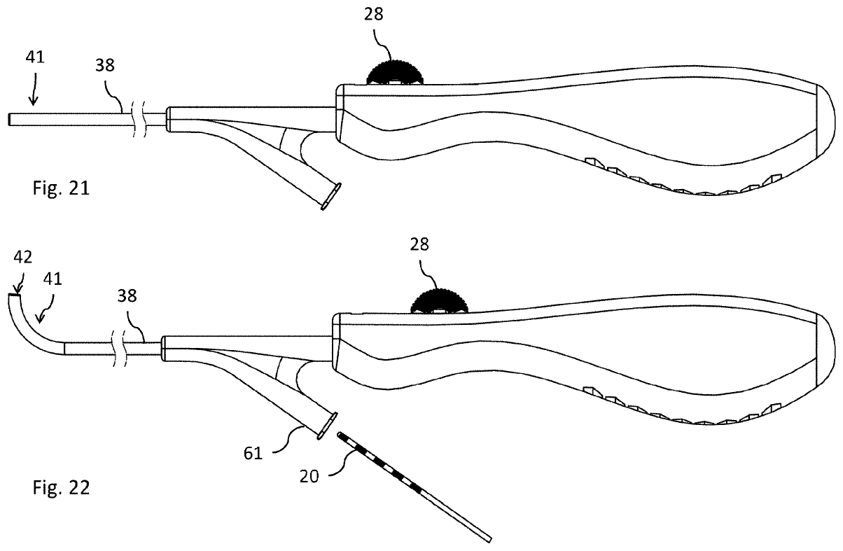

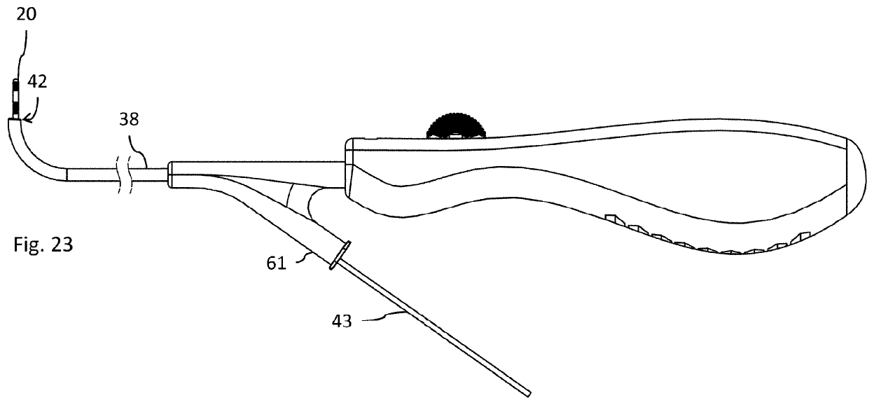

FIG. 21 shows the guide instrument 10 of FIG. 17 in the feed position in which the linear slide 28 is in a distal position, wherein the distal end region 41 of the feed tube 38 is coaxially aligned. During a proximal movement of the linear slide 28, the abutment 68 in contact with it is also moved in the proximal direction, whereby the pull wire 70 follows this movement. The pull wire 70 is, as already described, connected to the upper side 71 of the feed tube 38, so that the distal end region 41 of the feed tube 38 is raised by the movement of the linear slide 28, see FIG. 22, which shows the guide instrument 10 in the insertion position. The electrode 20 introduced into the connection piece 61 will, with continued axial movement, finally emerge from the distal end face 42 of the feed tube 38, as shown in FIG. 23.

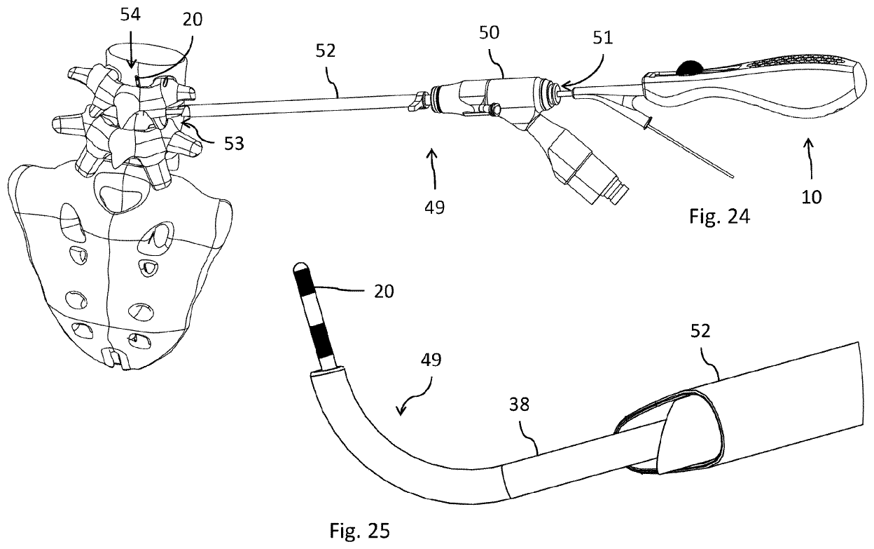

FIG. 24 shows, similar to FIG. 14, the device 49 according to the invention with the guide instrument 10 in the example with the endoscope 50, the example of which has already been discussed in connection with FIG. 14. The guide instrument 10 is introduced into the working canal 51 of the endoscope 50 in a known manner, which is also evident from the detailed view of FIG. 25. There, the feed tube 38 emerges from the cannulated working sleeve 52, wherein the distal end region 41 is curved in accordance with the insertion position of the guide instrument 10. According to FIG. 24, the device 49 shown, similar to FIG. 14, is introduced through the intervertebral foramen 53 to position the electrode 20 in the spinal canal 54.

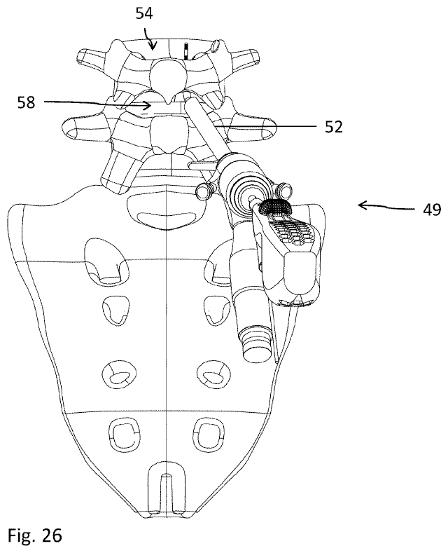

In FIG. 26, the device 49 according to the invention of FIG. 25 is arranged via the intralaminar access through the intralaminar window 58 in the spinal canal 54, similar to FIG. 16.

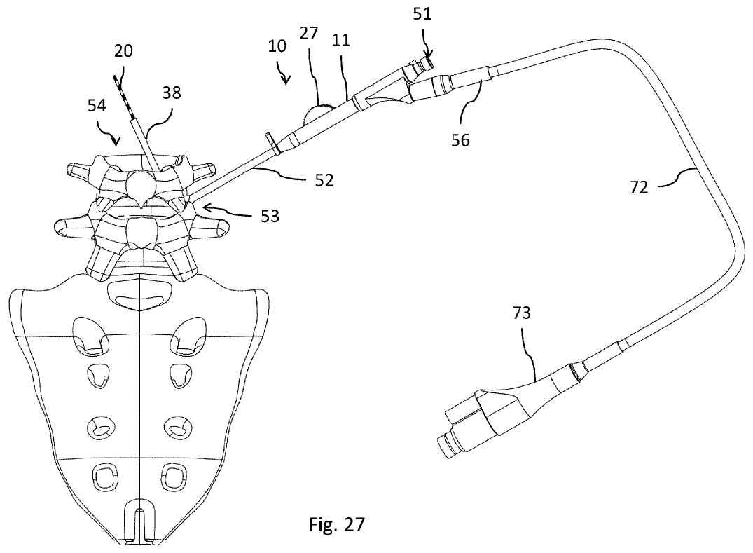

FIG. 27 shows an example of the medical guide instrument 10 according to the invention, which is inserted into the spinal canal 54 via the intervertebral foramen 53. Similar to the examples mentioned above, the guide instrument 10 of FIG. 27 has the distal feed tube 38, wherein its curved first portion 41 is not arranged as a distal end region, but in an axially central portion 41a. The axially central portion 41a is covered in perspective in FIG. 27. Due to the position of the first portion 41, a distal part of the feed tube 38 also engages in the spinal canal 54. The body part 11 is designed as a disposable endoscope, wherein the electrode 20, together with the line 43 and also the feed tube 38, are introduced via the coaxial working canal 51 of the endoscope. The optical output 56 is connected via a camera cable 72 with a connector 73 to a camera head cable. The working sleeve 52 of the endoscope enters the spinal canal 54 via the intervertebral foramen 53, similar to the illustration in FIG. 24. By actuating the actuator 27, it reaches the distal position shown in FIG. 27, so that a curvature of the first portion 41 of the feed tube 38—here arranged axially centrally—is bent and the electrode 20 can thus be arranged within the spinal canal 54 in the manner shown.

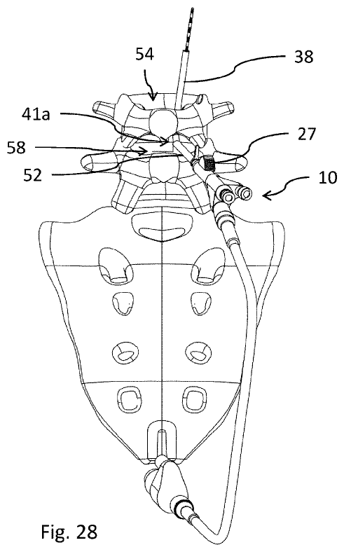

FIG. 28 shows the guide instrument 10 of FIG. 27, wherein access to the spinal canal 54 is achieved via the intralaminar window 58 already described. In FIG. 28, the axially central portion 41a of the feed tube 38 is also shown.

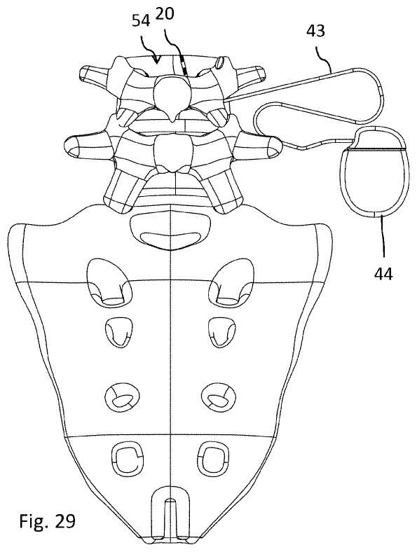

After the electrode 20 has reached the desired location in the spinal canal, in particular in contact with the dorsal nerve ganglion, and the effectiveness of the placement of the electrode 20 has been verified, the medical guide instrument 10 and the remaining components of the device 49 can be removed. The line 43 connected to the electrode 20 is then connected proximally to an electrical pulse generator 44 shown in FIG. 29, as already described in FIG. 13. The pulse generator 44 is designed to periodically and continuously output current pulses to the electrode 20 and can be implanted in the patient's body. For this purpose, the electrode 20 is positioned, for example, on a fascia with the aid of known anchoring devices, wherein strain relief loops ensure the stable hold of the electrode 20.

The method according to the invention will be described below using the example of the guide instrument 10 according to the invention in accordance with FIGS. 1 to 9 and 13 to 16:

First, access is created to the patient's spinal canal 54. This occurs, for example, via the intervertebral foramen 53 (FIG. 14) and/or via the intralaminar window 58 (FIG. 16). Alternatively or additionally, access can also be achieved transforaminally by means of puncturing a caudal part of the foramen with a spinal needle.

A guide wire is then placed, over which dilators with successively larger diameters are pushed one after the other in order to spread the tissue around the guide wire as atraumatically as possible. Finally, a working sleeve is introduced through the last dilator under constant X-ray control.

The working sleeve 52 of the endoscope 50 has an inner bore through which the endoscope 50 is introduced, wherein the endoscope 50 has, in addition to the flushing connector 57 and the optical output 56, the working canal 51 for receiving the guide instrument 10 according to the invention. The guide instrument 10 is pushed into the feed position through the working canal 51 of the endoscope 50 until its distal end region 41 engages in the spinal canal 54. The first portion 41 of the feed tube 38 of the guide instrument 10 is designed linearly to allow for rapid insertion into the patient's body.

After the guide instrument 10 has reached the desired location, the user actuates the actuator 27, i.e., pushes the linear slide 28 from proximal to distal, whereupon the feed tube 38 is also pushed in the distal direction, so that the first portion 41 of the feed tube 38 projects beyond the rigid guide tube 36 and, due to its material properties, assumes the curved position already described; the guide instrument is now in the insertion position.

In the next step, the electrode 20 together with the line 43 is inserted from the proximal side into the guide instrument 10, more precisely into its adapter part 19, until the electrode 20 emerges at the distal end face 42 of the curved feed tube 38 and can thereby assume the desired position, where it is brought into contact with the dorsal nerve ganglion in the spinal canal 54, for example. After the electrode 20 has assumed the desired position and its correct function has been checked, the guide instrument 10 together with the endoscope 50 is retracted so that ultimately only the electrode 20 remains in the patient. The electrode 20 is connected proximally to an electrical pulse generator 44, which is finally implanted in the patient as already described.

The invention being thus described, it will be obvious that the same may be varied in many ways. Such variations are not to be regarded as a departure from the spirit and scope of the invention, and all such modifications as would be obvious to one skilled in the art are to be included within the scope of the following claims.

What is claimed is:

1. A medical guide instrument for a medical electrode for positioning the electrode on spinal nerves in a spinal canal, in particular on spinal nerve nodes, the guide instrument comprising:
a distal feed tube for receiving the electrode,
wherein, when the guide instrument is in an insertion position, a first portion of the feed tube has a curvature, and
wherein, when the guide instrument is in a feed position, the first portion of the feed tube is substantially linear.

2. The medical guide instrument according to claim 1, wherein, in the insertion position of the guide instrument, the curvature of the first portion of the feed tube is user-defined or user-defined variably or manually adjustable.

3. The medical guide instrument according to claim 1, wherein the first portion is arranged in a distal region of the feed tube and is designed to be flexible as a distal end region.

4. The medical guide instrument according to claim 1, wherein a change between the feed position and the insertion position of the guide instrument is carried out due to an displacement of the feed tube.

5. The medical guide instrument according to claim 1, wherein the guide instrument has an actuator via which the insertion position of the guide instrument is assumed.

6. The medical guide instrument according to claim 5, wherein the actuator is at least indirectly connected to the feed tube or directly and in a positive fit.

7. The medical guide instrument according to claim 1, wherein the guide instrument is automatically in the insertion position without external force.

8. The medical guide instrument according to claim 1, wherein the feed tube is surrounded at least in portions by a rigid guide tube.

9. The medical guide instrument according to claim 1, wherein the guide instrument has an inner bore for receiving the electrode, and wherein the inner bore extends over the entire axial length of the guide instrument.

10. The medical guide instrument according to claim 5, wherein the feed tube is connected to the actuator at least indirectly via a pull wire.

11. The medical guide instrument according to claim 1, wherein the guide instrument is designed as a disposable instrument.

12. The medical guide instrument according to claim 1, wherein the first portion of the feed tube is arranged in an axially central portion of the feed tube.

13. The medical guide instrument according to claim 1, wherein the guide instrument has a flattening on its outer surface.

14. The medical guide instrument according to claim 1, further comprising an adapter part with an inner bore for receiving the electrode, wherein the inner bore of the adapter part is radially widened towards the proximal end face and/or towards the distal end face of the adapter part.

15. A medical instrument set comprising:
the medical guide instrument according to claim 1; and
a medical electrode with at least one electrically conductive contact surface and with a line connected to the electrode.

16. The medical instrument set according to claim 15, wherein the line has at least one resilient portion and/or at least one rigid portion.

17. A medical device comprising:
a medical guide instrument according to claim 1; and
a medical endoscope,
wherein the guide instrument is adapted to be received in a working canal of the endoscope.

18. A medical method for arranging a medical electrode on spinal nerves via the medical guide instrument according to claim 1, the method comprising:
providing access to the spinal nerves;
inserting the medical guide instrument in the feed position for positioning the electrode on the spinal nerves;
activating the insertion position of the medical guide instrument; and
inserting the electrode through the medical guide instrument through its feed tube until the electrode is positioned on the nerves.
